(19) United States
(12) Patent Application Publication
(10) Pub. No.: US 2016/0243311 A1
(43) Pub. Date: Aug. 25, 2016

(54) SLIDING PLUNGER-PLUG AND SYRINGE DEVICE COMPRISING SUCH A PLUNGER-PLUG

(71) Applicant: APTAR STELMI SAS, Villepinte (FR)

(72) Inventors: Ghislain FOURNIER, La Rochelle (FR); Mickael SWAL, Chauconin Neufmontiers (FR)

(73) Assignee: APTAR STELMI SAS, Villepinte (FR)

(21) Appl. No.: 15/028,074

(22) PCT Filed: Oct. 7, 2014
(86) PCT No.: PCT/FR2014/052540

§ 371 Date: Apr. 8, 2016

(30) Foreign Application Priority Data
Oct. 9, 2013 (FR) 1359773

(51) Int. Cl. A61M 5/315; A61L 31/10

(52) U.S. Cl. CPC A61M 5/31513; A61L 31/10; A61M 5/31576; A61M 2207/00; B29C 33/44

(57) ABSTRACT

A stopper/piston, in particular made of elastomer, having a cylindrical body having a front axial end that is closed by a front wall. The cylindrical body includes an outside surface provided with at least one sealing profile, the front wall including a front axial outside surface that is provided with a coating, advantageously a film made of ethylene tetrafluoroethylene (ETFE). The front wall includes a sealing profile on its radially-outer edge, the front axial outside surface of the front wall including a deformation profile that is arranged radially inside the sealing profile of the front wall. The deformation profile is adapted to deform radially so as to make it easier to insert and/or to slide said stopper/piston into a syringe body.

The present invention relates to a stopper/piston and to a syringe device including such a stopper/piston.

Stopper/pistons for syringe devices are well known in the prior art. Before the syringe is actuated, the stopper/piston performs the function of a stopper, isolating the fluid contained in the syringe body, and then during actuation, it transforms into a piston, pushing the fluid out of the syringe body, generally through a needle. Generally, the syringe device includes an actuator member, such as a piston rod, that co-operates with said stopper/piston so as to move it in the syringe body during actuation. The stopper/piston thus needs to guarantee sealing, and it is thus generally made of rubber or some other similar elastomer. A drawback with that type of material relates to the risks of interaction between the material of the stopper/piston and the fluid to be dispensed, in particular during the storage stage, where such interaction might possibly spoil said fluid. In order to limit the risks of interaction, it has been proposed to coat the front surface of the stopper/piston, i.e. the surface in contact with the fluid while storing and while dispensing the fluid, with an appropriate coating. In particular, it has thus been proposed to have a thin film of ethylene tetrafluoroethylene (ETFE) on the front surface of the stopper/piston. This embodiment makes it possible to limit the risks of interaction between the material of the stopper/piston and the fluid, but the presence of the coating on the front surface stiffens said surface, and thus makes it more difficult to assemble and move the stopper/piston in the syringe body. More precisely, the presence of the front coating reduces the capacity of the front surface of the stopper/piston to deform, and thus makes it more difficult to insert it in the syringe body and to slide it therealong. There thus exists a problem of finding a good compromise between sealing that is sufficient for the stopper/piston once inserted in the syringe body, and a process of assembly and of actuation that is not too complicated or difficult.

In addition, co-operation between the stopper/piston and the actuator member, generally a piston rod, generally occurs by means of a screw thread that is provided in the inside surface of the stopper/piston and that co-operates with a corresponding thread that is formed on the leading end of the piston rod. That technique also complicates manufacture of the stopper/piston, in particular the unmolding stage, as a result of the large amount of back-drafts required in the mold for manufacturing the stopper/piston in order to mold the thread.

Documents WO 2006/021380, EP 1 180 377, EP 1 674 121, DE 100 06 560, WO 02/092312 describe prior-art devices.

An object of the present invention is to provide a stopper/piston that does not have the above-mentioned drawbacks.

In particular, an object of the present invention is to provide a stopper/piston that guarantees complete sealing during storage and during actuation, while enabling assembly and actuation to be simplified and reliable.

Another object of the present invention is to provide a stopper/piston that is simpler and thus less costly to manufacture and to assemble, in particular in the process of molding said stopper/piston and while the stopper/piston is being inserted into a syringe body.

The present invention thus provides a stopper/piston, in particular made of elastomer, comprising a cylindrical body having a front axial end that is closed by a front wall, said cylindrical body including an outside surface that is provided with at least one sealing profile, said front wall including a front axial outside surface that is provided with a coating, advantageously a film made of ethylene tetrafluoroethylene (ETFE), said front wall including a sealing profile on its radially-outer edge, said front axial outside surface of said front wall including a deformation profile that is arranged radially inside said sealing profile of said front wall, said deformation profile being adapted to deform radially so as to make it easier to insert and/or to slide said stopper/piston into a syringe body.

Advantageously, said hollow cylindrical body includes a plurality of sealing profiles, advantageously two.

Advantageously, said at least one sealing profile of said hollow cylindrical body is a radially-projecting bead.

Advantageously, said deformation profile is made in the shape of a peripheral groove that is formed in said front axial outside surface of said front wall, said groove advantageously being V-shaped or U-shaped.

Advantageously, said sealing profile of said front wall is formed between said deformation profile and a radially-outer peripheral recess that is axially offset towards the rear relative to said front axial outside surface, said radially-outer peripheral recess also being deformed while said stopper/piston is being inserted into a syringe body and/or while it is being actuated. Advantageously, said cylindrical body is hollow and defines an internal volume, said hollow cylindrical body having an open rear axial end that is axially remote from said front axial end that is closed by said front wall.

Advantageously, said internal volume is adapted to co-operate with an actuator member of a syringe device.

Advantageously, on its inside surface, said hollow cylindrical body includes a screw thread that is adapted to be engaged on a thread of said actuator member.

Advantageously, said thread is discontinuous, a plurality of axial grooves, advantageously four grooves, made in the inside surface of said hollow cylindrical body, being provided so as to interrupt said thread.

Advantageously, said front axial outside surface is conical and defines a central axial tip.

In a variant, said front axial outside surface is substantially plane and perpendicular to a central axis of said stopper/piston.

Advantageously, said deformation profile is of rounded shape, such that said front axial outside surface of said front wall does not have any sharp angle.

The present invention also provides a syringe device including a cylindrical syringe body containing a fluid, said syringe body slidably receiving a stopper/piston as described above.

Advantageously, said at least one sealing profile of said hollow cylindrical body and said sealing profile of said front wall co-operate in leaktight manner with the cylindrical inside surface of said syringe body, said front axial outside surface being in contact with said fluid.

Advantageously, said sealing profile is provided, at least in part, with said coating, thereby defining a cylindrical sealing zone between the cylindrical inside surface of said syringe body and said coated sealing profile.

The present invention also provides a machine for manufacturing a stopper/piston as described above, including a first mold portion and a second mold portion, said first mold portion defining the shape of said hollow cylindrical body, and said second mold portion defining the shape of said front wall.

Advantageously, said first mold portion includes a core comprising a body that is extended by an axial endpiece defining said internal volume, said axial endpiece being provided with an external thread that is separated by a plurality of axial splines, advantageously four splines, defining a thread and axial grooves of said stopper/piston.

These characteristics and advantages and others of the present invention appear more clearly from the following detailed description, given by way of non-limiting example, and with reference to the accompanying drawings, in which:

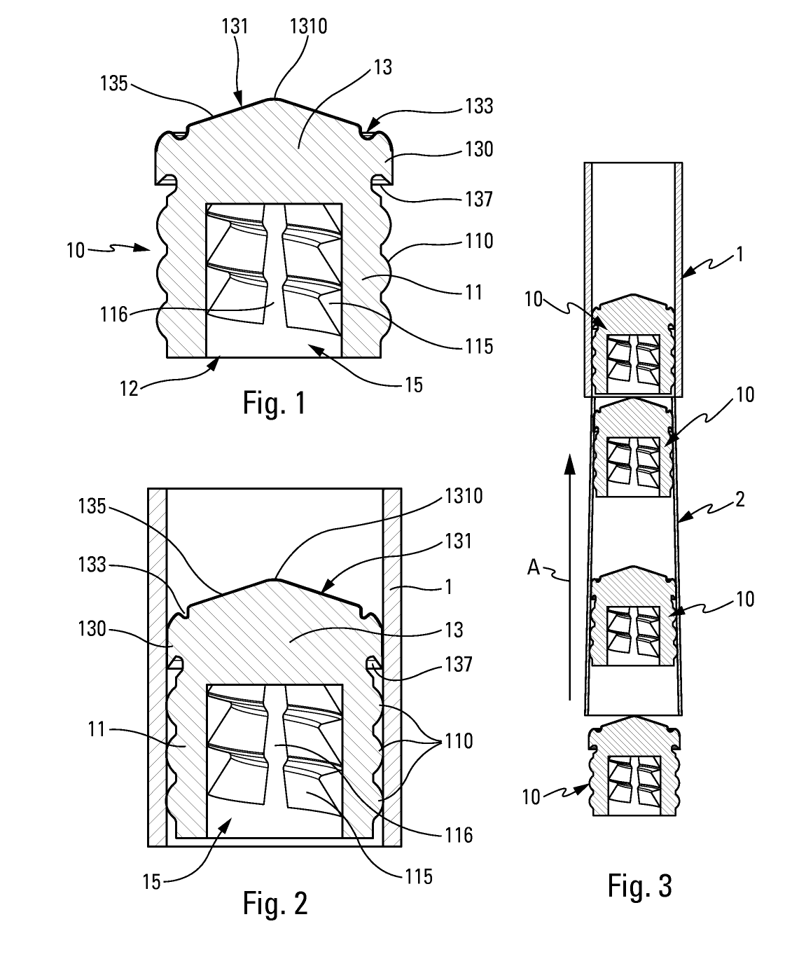

FIG. 1 is a diagrammatic section view of a stopper/piston in a first advantageous embodiment of the present invention;

FIG. 2 is a view similar to the view in FIG. 1, showing a stopper/piston after it has been inserted into a syringe body;

FIG. 3 is a diagram showing the various steps of the method of inserting the FIG. 1 stopper/piston in a syringe body;

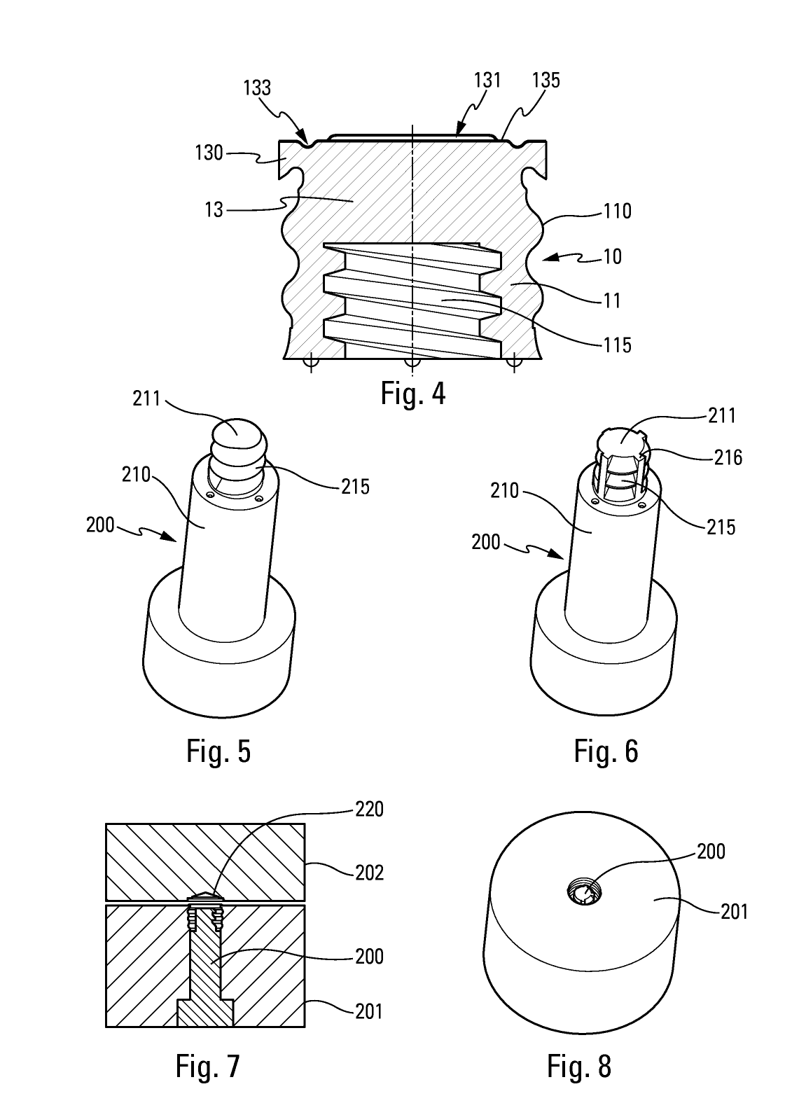

FIG. 4 is a view similar to the view in FIG. 1, showing a second advantageous embodiment of the present invention;

FIG. 5 is a diagrammatic perspective view of a mold core in a first variant embodiment;

FIG. 6 is a view similar to the view in FIG. 5 showing a mold core that is suitable for manufacturing a stopper/piston in a second variant embodiment;

FIG. 7 is a diagrammatic section view of the two mold portions used to manufacture the FIG. 1 stopper/piston;

FIG. 8 is a diagrammatic plan view in perspective of a FIG. 7 mold portion;

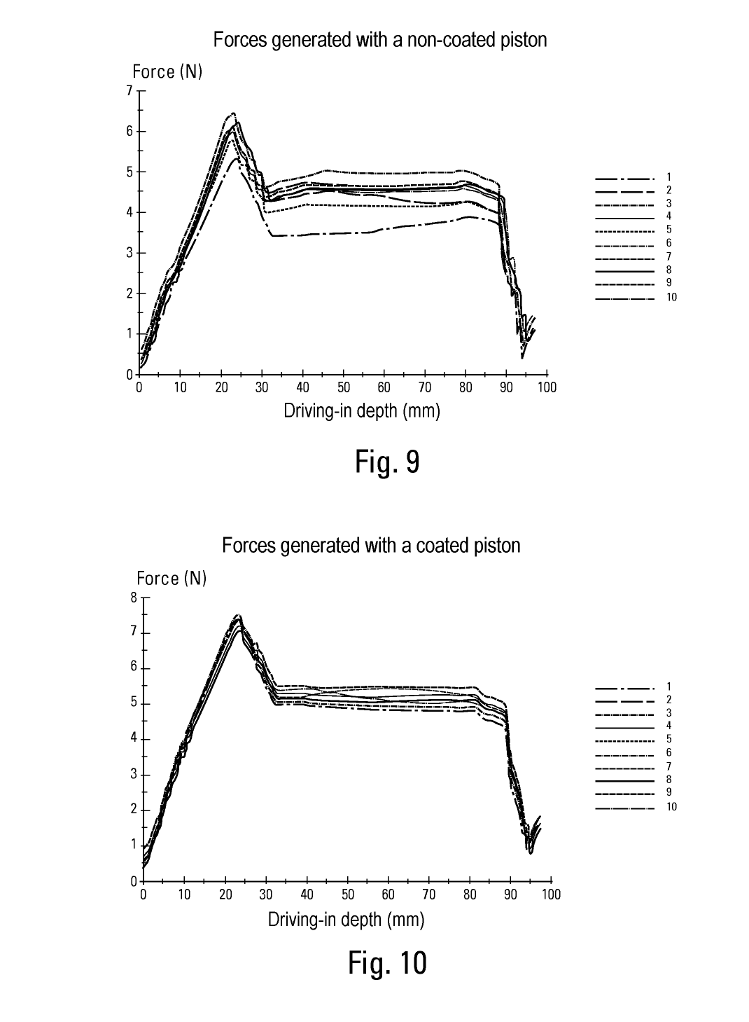

FIGS. 9 and 10 are graphs that compare the forces generated by pistons sliding in tubes, respectively without ETFE front coating and with ETFE front coating;

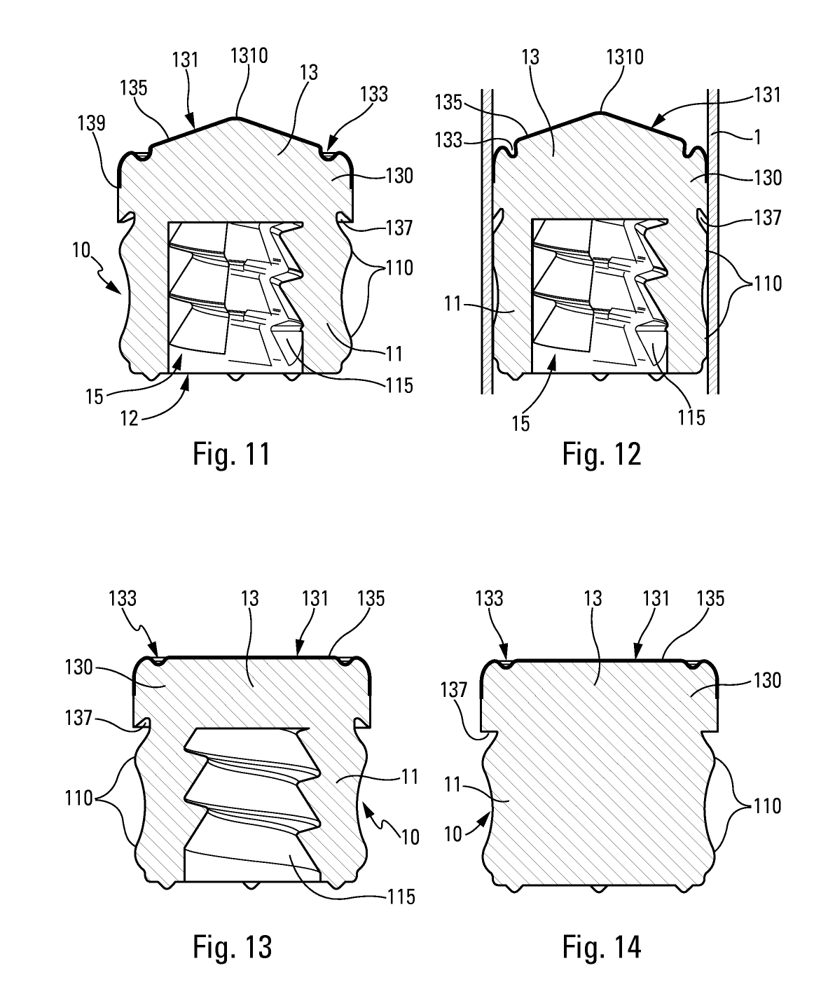

FIGS. 11 and 12 are views similar to the views in FIGS. 1 and 2, showing a variant embodiment with a conical front surface; and

FIGS. 13 and 14 are views similar to the views in FIG. 4, showing two variant embodiments with a plane front surface.

FIGS. 1 to 3 show a first embodiment of the invention. In this first embodiment, the stopper/piston 10, made of elastomer, rubber, or any other appropriate material, comprises a hollow cylindrical body 11. The hollow cylindrical body includes an open rear axial end 12, and a front axial end that is closed by a front wall 13. Thus, this embodiment defines a stopper/piston 10 of shape that is generally cylindrical, with an internal volume 15 that is blind, i.e. open at one end and closed at the other end. The front wall 13 defines a front axial outside surface 131 that is the surface that is in contact with the fluid when the stopper/piston 10 is assembled in a syringe body 1 for containing such a fluid. The front axial outside surface 131 is provided with a coating 135, advantageously a thin film made of ETFE, so as to limit the interaction between said fluid and the material of said stopper/piston as much as possible. Other types of appropriate coating could also be envisaged, both with regard to the method of applying the coating and with regard to the material constituting said coating. The front axial outside surface 131 may be conical, defining an axial tip 1310, as can be seen in the embodiments in FIGS. 1 to 3, 11, and 12, but it may also be plane and substantially perpendicular to the central axis of the stopper/piston, as shown in the FIGS. 4, 13, and 14.

The outside surface of the hollow cylindrical body 11 includes at least one sealing profile 110, and preferably a plurality of them that are generally made in the shape of radially-projecting beads. Preferably, and as shown in FIGS. 11 and 12, the outside surface of the hollow cylindrical body 11 includes two sealing profiles 110 that are made in the shape of radially-projecting beads. The front wall 13 also includes a sealing profile 130 that is made on its radially-outer edge.

In the present invention, said front axial outside surface 131 of said front wall 13 includes a deformation profile 133 that is adapted to deform so as to make it easier to insert the stopper/piston 10 into a syringe body 1. The deformation profile is arranged radially inside said sealing profile 130, i.e. it is not arranged at the radially-outer edge of said front wall 13. Advantageously, the deformation profile 133 is made in the shape of a peripheral groove that is formed in said front axial outside surface 131. In the embodiment shown in FIG. 1, before being deformed, the groove is substantially U-shaped, but obviously it could also have some other shape, e.g. a V shape, a W shape, or any other appropriate shape that enables the front wall 13 to deform radially while the stopper/piston is being inserted.

Advantageously, said deformation profile 133 is of rounded shape, i.e. it does not define a sharp angle in said front axial outside surface 131 of said front wall 13. This is because such sharp angles might generate zones of weakness in the coating 135, not only while molding the stopper/piston, during which they might generate portions that become torn off, but also in use during which the coating 135 might crack or tear at said sharp angles, e.g. while compressing the stopper/piston when putting it in place in the syringe, with the consequent risk of said coating being discontinuous.

Advantageously, said coating 135 extends not only over said front axial outside surface 131, but also over at least a part of the outside cylindrical surface of said sealing profile 130, as can be seen more clearly in

FIGS. 11 and 12. Advantageously, the coating 135 extends until it reaches a shoulder 139 of said sealing profile 130. This makes it possible to guarantee the presence of a first cylindrical sealing zone between the inside of the syringe body 1 and the coated portion of said sealing profile 130 of the stopper/piston 10. In particular, this improves sealing while high pressure is being exerted in the syringe. Specifically, a cylindrical contact zone between the syringe body 1 and the coated zone of the stopper/piston 10 guarantees the absence of micro-wrinkles in the coating 135, and thus makes it possible to have a sealing zone with contact that is constant between the coating 135 of the stopper/piston and the syringe body 1.

FIG. 2 shows the stopper/piston 10 after it has been inserted into a syringe body 1. It should be observed that the deformation profile 133 is deformed radially, and this enables the sealing profile 130 of the front wall 13 to penetrate more easily inside the syringe body 1.

FIG. 3 shows the various steps of the insertion process. At the bottom of FIG. 3, there can be seen the stopper/piston 10 arranged outside an insertion cone 2, with a deformation profile that is not deformed. The stopper/piston 10 is then inserted progressively inside said insertion cone 2 in the direction of arrow A, and as it advances inside said insertion cone, its front wall 13 deforms radially until it reaches maximum deformation, with which it may be inserted easily inside the syringe body 1, shown at the top of FIG. 3. After inserting said stopper/piston 10 into the syringe body 1, the various sealing profiles, i.e. the sealing profile 130 of the front wall 13 and the sealing profiles 110 of the hollow cylindrical body 11 may relax a little, while remaining sufficiently constrained against the cylindrical wall of the syringe body 1, so as to guarantee complete sealing.

The presence of the deformation profile 133 in the front axial outside surface 131 of the front wall 13 makes insertion easier. Specifically, the presence of the coating 135 stiffens the material and thus generally makes it more difficult to insert. FIGS. 8 and 9 demonstrate that the presence of an ETFE coating on the front axial outside surface 131 of the piston substantially increases the force necessary to move said piston in the insertion tube 2. Thus, an increase in the activation and sliding forces in the range about 30% to 40% is observed to the detriment of the piston with film. Thus, an activation force passes from about 6 newtons (N) without coating to about 8 N for a piston with coating, and the sliding forces pass from about 4 N to about 5.5 N. Provision of a deformation profile thus makes it possible to compensate for the increase in friction associated with the ETFE coating, and thus makes it possible to guarantee that the coated stopper/piston is inserted in more reliable and safe manner, as described above. In addition, the presence of the deformation profile 133 in the front axial outside surface 131 of the front wall 13 of the piston with coating makes it possible to have behavior that is identical to the behavior of a piston without coating, in particular with regard to the activation and sliding forces applied to the piston during actuation.

Advantageously, as can be seen in FIGS. 1 and 4 and 11 to 14, the sealing profile 130 of the front wall 13 is formed between said deformation profile 133 of the front axial outside surface 131 and a radially-outer peripheral recess 137. The radially-outer peripheral recess 137 is axially offset towards the rear relative to said front axial outside surface 131, and in FIGS. 2 and 12 it should be observed that during insertion of the stopper/piston into the syringe body 1, and subsequently, the radially-outer peripheral recess 137 is also deformed, in particular radially. This embodiment further facilitates the processes of insertion and of actuation, while guaranteeing complete sealing after insertion. Specifically, said sealing profile 130 of the front wall is urged radially outwards, and thus against the syringe body 1, both by the deformation profile 133 that has deformed elastically, and by the radially-outer peripheral recess 137 that has also deformed elastically.

The internal volume 15 of the stopper/piston 10 may include a thread 115. Advantageously, the thread 115 is discontinuous. This embodiment facilitates the process of molding the stopper/piston, as described below. Advantageously, a plurality of axial grooves 116, advantageously four grooves, are provided so as to interrupt said thread 115. With reference to FIG. 6, which shows the core of the mold that makes it possible to define the hollow cylindrical body 11 of the FIG. 1 stopper/piston 10, it should be observed that the endpiece 211 of the core 200 is provided with an external thread 215 that is interrupted or separated by four axial grooves 216. Thus, compared to the FIG. 5 core 200 in which the thread 215 is continuous, the FIG. 6 core makes it possible to make the interrupted thread of FIG. 1, which makes it easier, in particular, to unmold the stopper/piston by reducing back-draft.

Naturally, the embodiments in FIGS. 4 and 13, with the stopper/piston 10 having a front surface that is plane and perpendicular to the central axis, could also be made with such a discontinuous thread, if necessary.

FIG. 14 shows a stopper 10 having a front surface that is plane and perpendicular to the central axis, and having no thread.

FIG. 7 shows a machine for manufacturing the FIG. 1 stopper/piston, with a first mold portion 201 that is the bottom portion in FIG. 7, and a second mold portion 202 that is the top portion. The first mold portion 201 is used to form the hollow cylindrical body 11, and includes a core 200 for this purpose, as shown in FIG. 6. The core 200 comprises a body 210 that is extended by an axial endpiece 211 that defines said internal volume 15, and that thus includes said above-described discontinuous external thread 215 that is separated by the four axial splines 216. FIG. 8 is a perspective view from above of said first mold portion 201 which shows the core 200 inside the first mold portion 201.

Naturally, any number of axial splines 216 could be provided. If so desired, the FIG. 5 core could be used with a conical stopper/piston of FIG. 1, thereby making it possible to make a continuous thread in the internal volume 15.

In known manner, the piston of the invention may also be coated entirely with silicone oil so as to enhance its sliding, in particular while it is being inserted into the syringe. In a variant, when it is desired to omit silicone oil, a parylene coating may alternatively be provided, likewise over the entire piston. In this configuration, the ETFE film on the front surface of the piston could be treated so as to provide grip for the parylene.

Although the present invention is described above with reference to various embodiments, naturally the present invention is not limited by those embodiments, and, on the contrary, any useful modification could be applied thereto by the person skilled in the art, without going beyond the ambit of the present invention, as defined by the accompanying claims.

1. A stopper/piston, comprising a cylindrical body having a front axial end that is closed by a front wall, said cylindrical body including an outside surface that is provided with at least one sealing profile, said front wall including a front axial outside surface that is provided with a coating, advantageously a film made of ethylene tetrafluoroethylene (ETFE), said front wall including a sealing profile on its radially-outer edge, the stopper/piston being characterized in that said front axial outside surface of said front wall includes a deformation profile that is arranged radially inside said sealing profile of said front wall, said deformation profile being adapted to deform radially so as to make it easier to insert and/or to slide said stopper/piston into a syringe body.

2. A stopper/piston according to claim 1, wherein said hollow cylindrical body includes a plurality of sealing profiles, advantageously two.

3. A stopper/piston according to claim 2, wherein said at least one sealing profile of said hollow cylindrical body is a radially-projecting bead.

4. A stopper/piston according to claim 1, wherein said deformation profile is made in the shape of a peripheral groove that is formed in said front axial outside surface of said front wall, said groove advantageously being V-shaped or U-shaped.

5. A stopper/piston according to claim 1, wherein said sealing profile of said front wall is formed between said deformation profile and a radially-outer peripheral recess that is axially offset towards the rear relative to said front axial outside surface, said radially-outer peripheral recess also being deformed while said stopper/piston is being inserted into a syringe body and/or while it is being actuated.

6. A stopper/piston according to claim 1, wherein said cylindrical body is hollow and defines an internal volume (15), said hollow cylindrical body having an open rear axial end that is axially remote from said front axial end that is closed by said front wall.

7. A stopper/piston according to claim 6, wherein said internal volume co-operates with an actuator member of a syringe device.

8. A stopper/piston according to claim 7, wherein, on its inside surface, said hollow cylindrical body includes a screw thread that is adapted to be engaged on a thread of said actuator member.

9. A stopper/piston according to claim 8, wherein said thread is discontinuous, a plurality of axial grooves, advantageously four grooves, made in the inside surface of said hollow cylindrical body, being provided so as to interrupt said thread.

10. A stopper/piston according to claim 1, wherein said front axial outside surface is conical and defines a central axial tip.

11. A stopper/piston according to claim 1, wherein said front axial outside surface s substantially plane and perpendicular to a central axis of said stopper/piston.

12. A stopper/piston according to claim 1, wherein said deformation profile is of rounded shape, such that said front axial outside surface of said front wall does not have any sharp angle.

13. A syringe device including a cylindrical syringe body containing a fluid, the device being characterized in that said syringe body slidably receives a stopper/piston according to claim 1.

14. A device according to claim 13, wherein said at least one sealing profile of said hollow cylindrical body and said sealing profile of said front wall co-operate in leaktight manner with the cylindrical inside surface of said syringe body, said front axial outside surface being in contact with said fluid.

15. A device according to claim 14, wherein said sealing profile is provided, at least in part, with said coating, thereby defining a cylindrical sealing zone between the cylindrical inside surface of said syringe body and said coated sealing profile.

16. A machine for manufacturing a stopper/piston according to claim 1, characterized in that it includes a first mold portion and a second mold portion, said first mold portion defining the shape of said hollow cylindrical body, and said second mold portion defining the shape of said front wall.

17. A machine according to claim 16, wherein said first mold portion includes a core comprising a body that is extended by an axial endpiece defining said internal volume, said axial endpiece being provided with an external thread that is separated by a plurality of axial splines, advantageously four splines, defining a thread and axial grooves of said stopper/piston.

18. A stopper/piston according to claim 1, wherein the stopper/piston is made of elastomer.
